(19) United States
(12) Patent Application Publication
(10) Pub. No.: US 2020/0214764 A1
(43) Pub. Date: Jul. 9, 2020

(54) DEVICES, SYSTEMS AND METHODS FOR ACCESSING A BODY LUMEN

(71) Applicant: Boston Scientific Scimed, Inc., Maple Grove, MN (US)

(72) Inventors: Evan Wilder, Boston, MA (US); Peter L. Dayton, Brookline, MA (US); Douglas Melanson, Natick, MA (US)

(21) Appl. No.: 16/732,477

(22) Filed: Jan. 2, 2020

Related U.S. Application Data
(60) Provisional application No. 62/787,820, filed on Jan. 3, 2019.

(51) Int. Cl. A61B 18/14

(52) U.S. Cl. CPC A61B 18/1492; A61B 2018/00601

(57) ABSTRACT

The present disclosure relates generally to the field of medical devices for accessing body lumens. In particular, the present disclosure relates to medical devices, systems and methods for targeted access to and cutting of a body lumen opening with an electrode. In embodiments, a device may comprise a flexible elongate tube, an articulation wire may extend at least partially along the tube within a lumen from the proximal end of the tube and have a distal end connected to a distal portion of the elongate tube, a portion of the articulation wire may extend external to the elongate tube, an electrode may be disposed on the distal portion of the tube distal to the distal end of the articulation wire, and a conductive wire may extend along and within the tube in electrical communication with the electrode.

### PRIORITY

This application claims the benefit of priority under 35 USC § 119 to U.S. Provisional Patent Application Ser. No. 62/787,820, filed Jan. 3, 2019, which is incorporated by reference herein in its entirety and for all purposes.

### FIELD

The present disclosure relates generally to the field of medical devices for accessing a body lumen. In particular, the present disclosure relates to medical devices, systems and methods for targeted access to and cutting of a body lumen opening with an electrode.

### BACKGROUND

Medical professionals sometimes face significant technical challenges when accessing a body lumen, such as when performing endoscopic cannulation procedures that may require advancing a guidewire and/or endoscopic accessory tool (e.g., sphincterotome, cannula, catheter, or the like) against, into, or through challenging patient anatomies. For example, endoscopic retrograde cholangiopancreatography (ERCP) may be performed by trained gastroenterologists and surgical endoscopists to diagnose and treat various disorders of the pancreaticobiliary system. Physicians in such procedures may use a sphincterotome to cannulate a body lumen (e.g., the papillary orifice or the like). A sphincterotome may include a guidewire, so that after initial cannulation the guidewire can be inserted into the biliary duct. A sphincterotome may include a cutting wire for cutting/accessing tissue of an opening (e.g., a sphincterotomy or the like). The end of a sphincterotome may have a flexible distal end that may be articulated (e.g., bowed) by articulating the cutting wire. The distal end of the sphincterotome may need to be extended into the opening of the body lumen, so that the cutting wire may reach the tissue of the opening for cutting. However, if access to and/or cannulation of the body lumen is difficult or impossible, the opening or access point into a body lumen may need to be precut (e.g., a precut sphincterotomy or the like) before cannulating. This may require the use of a needle-knife having a distal electrode for cutting into an opening of body lumen. A needle-knife may not include a flexible end capable of being articulated to control the orientation and shape of the instrument. The distal electrode of a needle-knife may cut open an access point or opening to the body lumen, and the needle-knife may then be withdrawn from the patient and a sphincterotome may be inserted and used to further access and/or cannulate the body lumen opening.

A variety of advantageous medical outcomes may be realized by the medical devices, systems, and methods of the present disclosure, which integrate targeted access to and cutting of body lumens in challenging patient-specific anatomies.

### SUMMARY

Embodiments of the present disclosure may assist generally with accessing and/or cutting of a body lumen opening without the need to use multiple devices and/or devices that do not combine maneuverability and a controlled cutting electrode. In one aspect, a medical device may include a flexible elongate tube that may have a proximal end, a distal end, an outer surface, and a distal portion proximal to the distal end. The distal end may be configured to access an opening of a body lumen. A first lumen may extend from the distal end of the tube at least partially along the tube toward the proximal end of the tube. The first lumen may be configured to accept a guidewire. A second lumen may extend at least partially along the tube from the proximal end of the tube. An articulation wire may extend along the second lumen. A distal end of the articulation wire may be connected to the distal portion of the elongate tube. A portion of the articulation wire, which may be proximal to the distal end of the articulation wire, may extend external to the outer surface of the elongate tube. An electrode may be disposed on the distal portion of the tube distal to the distal end of the articulation wire. A conductive wire may extend along and within the tube. The conductive wire may be in electrical communication with the electrode. The electrode may extend linearly on the outer surface of the tube from the distal portion to an opening of the first lumen at the distal end of the tube. The electrode may extend in a loop on the outer surface of the tube from the distal portion around the first lumen. The electrode may comprise 304 stainless steel, aluminum, silver, or titanium. The electrode may be a separate element disposed on the outer surface of the tube along the distal portion and coupled to the conductive wire. A third lumen may extend from the proximal end along the elongate tube and may be configured to deliver a contrast agent to the distal end of the tube. An anchor may be within the distal portion of the tube and may be coupled to the distal end of the articulation wire. The second lumen may extend to the distal portion of the tube. The anchor may be disposed within the second lumen. A handle may be at the proximal end of the elongate tube and may be connected to the articulation wire. The handle may be actuatable to slide the articulation wire within the second lumen and articulate the distal end of the tube toward the opening of the body lumen. The articulation wire proximal and distal to the external portion may be disposed within the second lumen. The second lumen may extend to the distal portion. The articulation wire may be slidably translatable within the second lumen to articulate the distal end of the tube. The articulation wire and the conductive wire may be the same wire. The electrode may have a surface area on the outer surface of the tube along the distal portion and may have a circle, oval, square, or rectangular shape. The shape may be disposed on the outer surface by printing, painting, etching, molding, adhering, welding or soldering.

In another aspect, a medical device may include a flexible elongate tube that may have a proximal end, a distal end, an outer surface, and a distal portion proximal to the distal end. The distal end may be configured to access an opening of a body lumen. A first lumen may extend from the distal end of the tube at least partially along the elongate tube toward the proximal end of the elongate tube. The first lumen may be configured to accept a guidewire. A second lumen may extend at least partially along the tube from the proximal end of the tube. An electrode may be disposed on the distal portion of the tube. An articulation wire may extend along the second lumen. A distal end of the articulation wire may be in electrical communication with the electrode. A portion of the articulation wire, which may be proximal to the distal end of the articulation wire, may extend external to the outer surface of the elongate tube. An insulative layer may be disposed on the external portion of the articulation wire. An anchor may be disposed in the second lumen along the distal portion. The anchor and the electrode may be a continuous unibody. The articulation wire may be electrically coupled to the anchor. The electrode may extend linearly on the outer surface of the tube from the distal portion to an opening of the first lumen at the distal end of the tube. The electrode may extend in a loop on the outer surface of the tube from the distal portion around the first lumen.

In another aspect, a method of accessing an opening of a body lumen may include inserting an elongate tube of a medical device that may have a distal end into a patient to the opening of the body lumen. The distal end of the elongate tube may be articulated toward the opening of the body lumen via an articulation wire extending through the elongate tube. The wire may be connected at the distal end of the tube and may have a portion external to an outer surface of the elongate tube that may be slidable relative to the tube to articulate the distal end toward the opening. An electrode on the outer surface at the distal end of the elongate tube may be activated to cut tissue of the opening. The electrode may be an extension of the articulation wire. The electrode may be a separate element in electrical communication with a conductive wire extending along the tube. The distal end of the elongate tube may be oriented such that a portion of the electrode makes contact with the tissue of the body lumen. A contrast agent may be delivered into the body lumen.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1A illustrates a conventional endoscopic system and accessory device accessing the papilla of a patient through the stomach and the duodenum.

FIG. 1B illustrates a closer view of the papilla and surrounding system and tissue of FIG. 1A.

FIG. 2 illustrates an exemplary device for accessing a body lumen, according to an embodiment of the present disclosure.

FIG. 3 illustrates an exemplary device for accessing a body lumen, according to an embodiment of the present disclosure.

FIG. 4 illustrates an exemplary device for accessing a body lumen, according to an embodiment of the present disclosure.

FIG. 5 illustrates an exemplary device for accessing a body lumen, according to an embodiment of the present disclosure.

### DETAILED DESCRIPTION

The present disclosure is not limited to the particular embodiments described. The terminology used herein is for the purpose of describing particular embodiments only and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure may be described with specific reference to medical devices and systems (e.g., endoscopic devices, accessory tools, and/or guidewires inserted through a duodenoscope, near or through a papilla, or the like) for selective access to, aligning with, cannulation, and/or cutting of the opening to the common bile duct (CBD) or pancreatic duct (PD) during an ERCP, it should be appreciated that such medical devices and systems may be used in a variety of medical procedures which require navigating one or more accessory tools through ductal, luminal, vascular, or body lumen anatomies, including, for example, interventional radiology procedures, balloon angioplasty/angiography procedures, thrombolysis procedures, urological or gynecological procedures, and the like. The disclosed medical devices and systems may also be inserted via different access points and approaches, e.g., percutaneously, endoscopically, laparoscopically, or combinations thereof.

As used herein, the singular forms “a,” “an,” and “the” are intended to include the plural forms as well, unless the context clearly indicates otherwise. The terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

As used herein, the term “distal” refers to the end farthest away from the medical professional when introducing a medical device into a patient, while the term “proximal” refers to the end closest to the medical professional when introducing a medical device into a patient.

As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g. 1 to 5 includes 1, 1.5, 2, 2.75, 3, 3.80, 4, and 5).

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

Use of a flexible accessory device, such as a sphincterotome or the like, to attempt to cannulate an opening of a body lumen only to discover a complication that makes the task infeasible, overly complicated, or otherwise results in a lengthy procedure, may be detrimental to the patient. The flexible accessory device may need to be withdrawn from the patient in order to introduce a secondary device, such as a needle-knife, to cut open or otherwise access the body lumen. This secondary device may then need to be withdrawn to reintroduce the flexible accessory device, which may then be used to further cannulate the opening of the body lumen. Such a process may increase procedure time and agitation to tissue of the body lumen opening and/or other tissue of the body lumen.

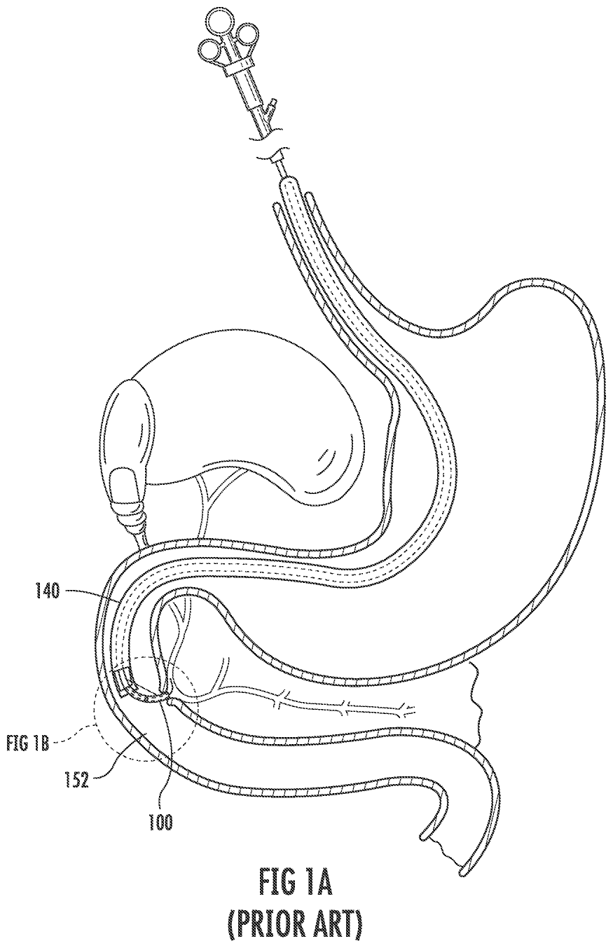

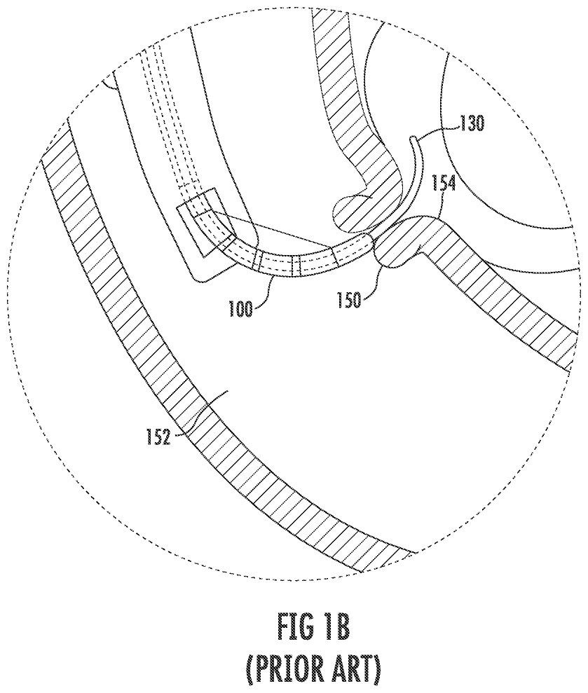

With reference to FIGS. 1A and 1B, a known process of selective cannulation during an ERCP procedure is illustrated, which includes a guidewire 130 and/or an endoscopic accessory device 100 being directed towards, against, and/or through a body lumen opening such as the major papilla 150 (e.g., ampullary entry) near the descending duodenum 152 to access the Sphincter of Oddi Complex 154. A distal portion of a medical device 140 (e.g., duodenoscope) may be positioned within the descending duodenum 152. The guidewire 130 and the medical device 100 may be advanced through a working channel of the duodenoscope 140 towards the major papilla 150. Additionally, the guidewire 130 and/or the medical device 100 may be advanced against or through the major papilla 150. Accessing the papilla 150 may be difficult because the opening is small compared to many medical devices, the opening may be completely collapsed/closed, and/or the opening may extend into the descending duodenum 152 at an angle that may be difficult to visualize and/or access the opening. Thus, a medical professional may be required to articulate the medical device 100 and guidewire 130 by manually rotating the duodenoscope 140, using an elevator within the distal end of the duodenoscope 140, and/or by articulating a cutting wire on the medical device 100 (e.g., sphincterotome) in an attempt to better align or orient the device 100 and/or guidewire 130 with respect to the duodenscope and the opening of the papilla. If access is difficult or impossible, a body lumen opening may need to be pre-cut (e.g., via a pre-cut sphincterotomy or the like) before cannulating. This may require the use of an additional medical instrument, such as a rigid needle-knife, having a distal electrode for cutting into the opening of the body lumen, with the attendant potential disadvantages mentioned above. Particularly difficult cannulation procedures in which the medical professional fails to access the Sphincter Papillae within a certain time limit, or after a certain number of unsuccessful attempts, may lead to significant post-procedure complications, such as post-ERCP pancreatitis (PEP).

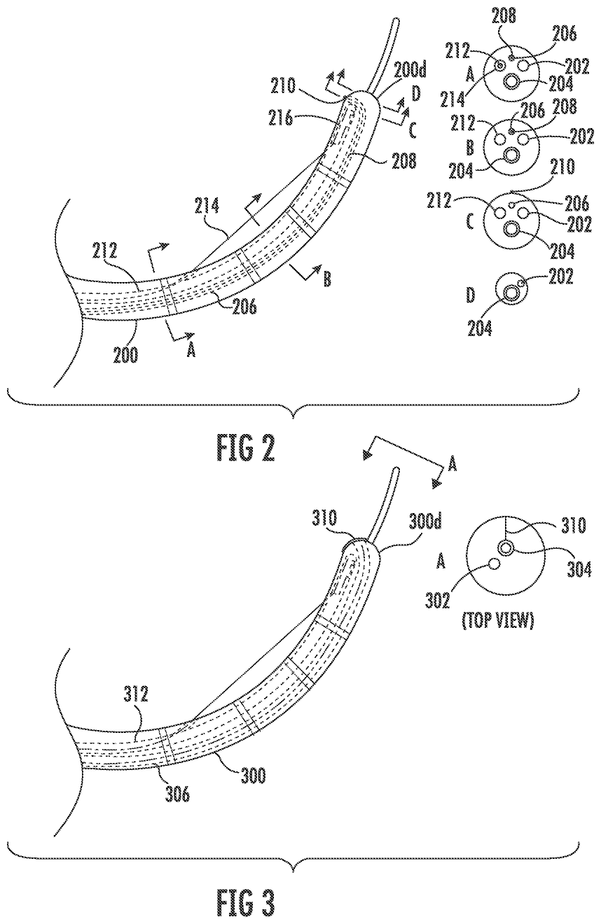

With reference to FIG. 2, an embodiment of a device for accessing a body lumen according to the present disclosure is illustrated, which includes a flexible elongate tube 200 with a distal end 202d configured to access an opening of a body lumen. A number of lumens may extend along the tube 200. As shown, a guidewire lumen 204 extends from the distal end 200d to the proximal end of the tube (i.e., from the cross-section D of the tube 200, through the cross-section C, through the cross-section B, and through the cross-section A). In embodiments, the guidewire lumen 204 is configured to accept a guidewire extending along the guidewire lumen 204, and may extend all the way to the proximal end of the device or may terminate at some point distal to the proximal end (e.g., at a proximal guidewire port when the device is configured to allow for rapid exchange of the guidewire). The guidewire may be used to guide the device and other instruments into the body lumen once the opening is accessed. An articulation wire lumen 212 extends from a distal portion of the tube 200 toward the proximal end of the tube 200 (i.e., from the cross-section C and through cross-sections B and A). An articulation wire 214 extends through the articulation wire lumen 212. The articulation wire 214 may be slidably translatable within the articulation wire lumen 212 to articulate the distal end 200d of the tube 200. A portion of the articulation wire 214 that is proximal to the distal end of the articulation wire 214 extends external to the articulation wire lumen 212 of the elongate tube 200. In some embodiments, the lumen 212 may terminate at a point proximal to the distal portion, e.g., at a point where the articulation wire 214 exits the elongate tube 200 and extends externally to an outer surface of the tube 200. A distal end of the articulation wire 214 is connected to the distal portion of the tube 200. The distal end of the articulation wire 214 is connected to an anchor 216 within the articulation wire lumen 212. In embodiments, an anchor 216 could be configured within the distal portion, but not within a lumen. The anchor 216 is distal to the portion of the articulation wire 214 that extends external to the articulation wire lumen 212. A handle (not shown) may be connected to the articulation wire 214. Proximal-tension, distal-pressure, and/or a release of tension on the articulation wire 214 (e.g., via the handle) may allow for the external portion of the articulation wire 214 to bow and/or otherwise be manipulated and may articulate the distal end 200d of the tube 200. An electrode 210 is disposed on the outer surface of the tube 200 along the distal portion of the tube 200 that is distal to the distal end of the articulation wire 214 at cross-section C. A conductive wire lumen 206 for a conductive wire 208 extends from the distal portion of the tube 200 toward the proximal end of the tube 200 (i.e., from cross-section C through cross-sections B and A). The conductive wire 208 extends along and within the conductive wire lumen 206 and is in electrical communication with the electrode 210 along the distal portion. As shown in FIG. 2, the electrode 210 is the end of the conductive wire 208, exposed of insulation at the point where the conductive wire 208 exits the conductive wire lumen 206 to the outer surface along the distal portion. In other embodiments, the conductive wire 208 may be coupled to a separate electrode element 210 on the outer surface, as described further below. A contrast lumen 202 extends from a proximal end to the distal end 200d along the elongate tube 200 (i.e., from cross-section D through cross-sections C, B, and A), and is configured to deliver a contrast agent. For clarity, hidden lines are used to illustrate the articulation wire lumen 212 and the conductive wire lumen 206, but hidden lines are not used to illustrate the guidewire lumen 204 and the contrast lumen 202.

In various embodiments, a flexible elongate tube of a device may comprise an extrusion of multiple lumens. The lumens may be extended through a proximal end of the tube to the distal end of the tube, even if the entire portion of the lumen will not contain a corresponding feature. One or more lumens (e.g., an articulation wire lumen) may terminate at a proximal exit point for a feature (e.g., an external portion of an articulation wire). The lumens may be configured to accept features (e.g., contrast, guidewire, articulating wire) of the device through at least a portion of the lumen. Such features may extend partially along a lumen and may extend external to the lumen along various portions of the lumen on an outer surface of the elongate tube. The feature may extend back within the lumen at another portion of the feature (e.g., an external portion of an articulation wire). Such features or portions of a feature extending external to a lumen may break through a wall of the lumen and/or tube such that they are external to the lumen on the outer surface of the tube. One or more ends of the tube may be drawn down and/or treated at the tip such that some of or all of the lumens are closed at an end. A drawn-down distal or proximal end may have a smaller diameter than a diameter of other portions of the tube.

With reference to FIG. 3, an embodiment of a device for accessing a body lumen according to the present disclosure is illustrated, which includes an electrode 310 extending from a distal portion of the flexible elongate tube 300 to the distal end of the elongate tube 300d, and substantially linear/radial to an opening of the guidewire lumen 304. This electrode 310 has a larger surface area than that of the electrode 210 in FIG. 2. The electrode 310 may be articulated to contact tissue along a line matching the shape of the electrode 310. The distal portion and distal end 300d of the tube 300 may be articulated such that the electrode 310 in the shape of a line sweeps across an arc about a longitudinal axis of the tube 300 and may cut a segment of tissue along the arc. In use, a guidewire or patient anatomy may obstruct vision of the electrode 310. However, a medical professional may continue operating the device knowing that the electrode is located at the guidewire lumen 304. Additionally, or in the alternative, the tube 300 may be articulated generally away from the longitudinal axis (e.g., substantially radially) so as to make a linear cut in a motion that is parallel to a length of the electrode 310. A contrast lumen 302 extends from a proximal end to the distal end 300d along the elongate tube 300 and is configured to deliver a contrast agent. For clarity, hidden lines are used to illustrate the articulation wire lumen and the conductive wire lumen, but hidden lines are not used to illustrate the guidewire lumen 304 and the contrast lumen.

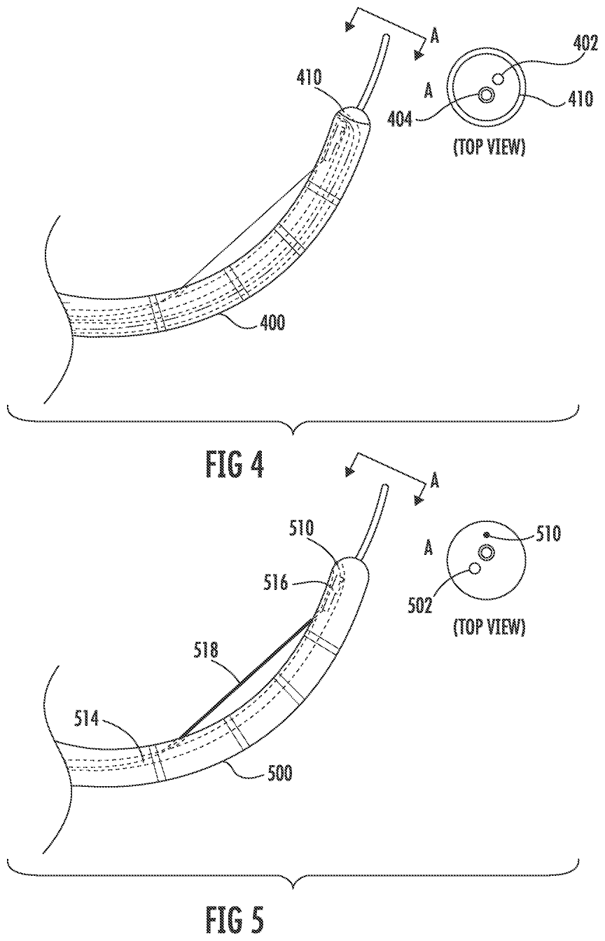

With reference to FIG. 4, an embodiment of a device for accessing an opening according to the present disclosure is illustrated, which includes an electrode 410 extending in a loop around a guidewire lumen 404 and a contrast lumen 402 at a distal portion of a flexible elongate tube 400. The distal portion of the tube 400 may be inserted into a body lumen such that the entire electrode 410 makes circumferential contact with the opening of the body lumen or the wall of the body lumen. Alternatively, the distal portion of the tube 400 may be articulated such that a longitudinal axis of the tube 400 is oriented to be at a desired angle to a longitudinal axis of the body lumen such that only a segment of the electrode 410 makes contact with the opening or wall of the body lumen. For clarity, hidden lines are used to illustrate the articulation wire lumen and the conductive wire lumen, but hidden lines are not used to illustrate the guidewire lumen 404 and the contrast lumen 402.

With reference to FIG. 5, an embodiment of a device for accessing a body lumen according to the present disclosure is illustrated, which includes an electrode 510 on a distal portion of a flexible elongate tube 500. A portion of an articulation wire 514 extends out of a articulation wire lumen, external to the outer surface of the tube 500. This external portion of the articulation wire 514 extends back into the tube along the distal portion and is in electrical communication with an anchor 516. A distal end of the articulation wire 514 extends from the anchor 516 to the electrode 510 and is in electrical communication with the electrode 510. The articulation wire is conductive and an insulative layer 518 is disposed on the portion of the articulation wire 514 that is external to the tube 500. The coupling of the distal end of the articulation wire 514 to the electrode 510 supplants the need for a separate conductive wire and separate conductive wire lumen for the electrode 510. The conductive articulation wire 514 provides an electrical connection for the electrode 510 and is able to energize the electrode. Additionally, or in the alternative, the articulation wire 514 may terminate at the anchor 516 and the anchor 516 may be exposed from the tube 500 to act as an/the electrode. Additionally, or in the alternative, the articulation wire 514 may extend past the anchor 516, with the portion of the articulation wire 514 extending past the anchor 516 being exposed as the electrode 510 at the distal portion of the tube 500 and/or couple to a separate electrode 510 on the outer surface of the tube 500. A contrast lumen 502 extends from a proximal end to the distal end along the elongate tube 500 and is configured to deliver a contrast agent. For clarity, hidden lines are used to illustrate the articulation wire lumen, but hidden lines are not used to illustrate the guidewire lumen and the contrast lumen.

In various embodiments, described here or otherwise within the scope of the present disclosure, a distal end of an electrically conductive articulation wire may extend to and terminate at an anchor. There may be no separate electrode disposed on the flexible elongate tube. A portion of the articulation wire that is external to the tube may include an insulative layer that is disposed on at least some of the portion of the manipulating wire that is external to the tube. For example, a portion of the articulation wire may be partially coated with aluminum oxide, PTFE, or the like. A distal segment of the articulation wire that may extend distally from the anchor may be exposed without an insulating layer. This may be achieved, for example, by coating the entire articulation wire and then stripping a portion of the wire of the coating to create an exposed segment of the wire. This exposed distal segment may be used as the electrode for cutting. An exemplary exposed segment of a wire may be about 0 mm to about 5 mm. An embodiment may include an insulated articulation wire with an exposed distal segment that may be used as an electrode in addition to a separate electrode disposed on the tube with its own conductive wire in electrical communication with the electrode. The electrode and the distal segment of the articulation wire may be separately activated and controlled during a procedure should either or both be needed. A conductive articulation wire may be separate and not electrically connected to the electrode, which may have its own conductive wire, so that each of the articulation wire and the electrode may be monopolar and independently activated. Alternatively, the conductive articulation wire and the electrode may be electrically connected to operate in a bipolar arrangement. An electrode may be a separate element disposed on an outer surface of the distal portion of a tube.

In various embodiments, an elongate flexible tube may comprise polytetrafluoroethylene (PTFE), fluorinated ethylene propylene (FEP), polyether ether ketone (PEEK), or the like, or a combination thereof. The tubes may be extruded to include one or more lumens described herein. An extruded tube may include the one or more lumens extending from a proximal end to the distal end even though a portion or an entirety of each lumen may not contain a wire, device, fluid, etc. For example, a conductive wire may extend substantially radially out of a lumen to an electrode proximal to a distal extension of the lumen, which may remain empty. For another example, an articulation wire may extend substantially radially out of a lumen at a point and the articulation wire may extend substantially radially back into the lumen at a more distal point (e.g., to an anchor), etc. Such lumens may be broken by a user (e.g., to expose an external portion of an articulation wire), include apertures for access and/or egress points, perforations, thin walls, etc. An articulation wire lumen may terminate where the articulation wire exits the tube at the proximal end of the external portion of the articulation wire. An anchor for the articulation in such an embodiment where the lumen does not extend to the distal portion of the tube may be disposed within the tube, but not within a lumen.

In various embodiments, the various lumens described within a flexible elongate tube may be arranged in different configurations and combinations depending on the instrument and requirements for a particular application. For example, a lumen configured for a guidewire may also be configured (or instead be configured) for a contrast agent, etc. Embodiments described and illustrated herein are not meant to exclusively include only those lumens, and do not necessarily need to include all of the lumens illustrated.

In various embodiments, an electrode or a portion of an articulation wire or a conductive wire used as an electrode may have a smaller surface area than the length of an exposed portion of a cutting wire in a conventional sphincterotome. A smaller surface area electrode may increase the power of the electrode with a high current density supplied with the same amount of current, when compared to a longer cutting wire of a conventional sphincterotome. A smaller surface area electrode may provide a physician with precise cutting control when compared to larger electrodes and/or cutting wires. A precision cutting electrode may reduce the risk of ancillary burns (e.g., duodenal burns or the like) and may improve the quality of tissue cuts.

An electrode that is a separate element coupled to a conductive wire or conductive articulation wire may be formed in a variety of shapes, e.g., a circle, an ellipsoid, a rectangle, a star, a triangle, a combination thereof, or the like. A segment of an exposed and electrified articulation wire that is not insulated may also be used as a smaller surface area electrode than that of a cutting wire of a conventional sphincterotome. A conductive wire, electrode, articulation wire, anchor, may comprise stainless steel, 304 stainless steel, gold, silver, tungsten, titanium, or the like, or a combination thereof. An electrode element may be in electrical communication with a conductive articulation wire and/or an anchor, or conductive wire separate from the articulation wire, by soldering, printing, painting, etching, welding, adhesion, molding, winding, tying, or the like. An electrode may be flush with an elongate tube or it may extend away from the outer surface of the elongate tube. An elongate tube may be articulated such that an extended electrode may only have an end point of the electrode in contact with tissue for treatment rather than the entire surface area of the extended electrode. An electrode may be, for example, about 0 mm to about 5 mm in length.

The devices, systems, and methods of the present disclosure may be used as sphincterotomes for cannulation, papillotomy, sphincterotomy, and the like. Exemplary devices, systems, and methods with which embodiments of the present disclosure may be implemented include, but are not limited to, those described in U.S. Pat. Nos. 6,676,659, 6,827,718, 7,371,237, 7,635,363, 8,231,621, 8,579,895, and 9,352,124, and U.S. patent application Ser. No. 15/158,052, each of which are herein incorporated by reference in their entirety. Exemplary devices described therein may be modified to incorporate embodiments or one or more features of the present disclosure. For example, a medical device may not include a guide wire or a contrast agent.

In various embodiments, described here or otherwise within the scope of the present disclosure, a lumen of the flexible elongate tube may be configured to receive a guidewire such that a distal portion of the guidewire may extend distally beyond the distal end of the elongate tube. The elongate tube may include a flexible distal portion and be configured to move with the articulation wire (e.g., flex, bend, rotate, wobble, spin, etc.) in a plurality of directions (e.g., x, y and/or z directions), thereby imparting an identical or similar direction of movement to the distal portion of the guidewire and/or an electrode. The distal portion of the elongate tube may be configured to move in a linear motion (e.g., moving in a single direction along a straight line relative to a longitudinal axis of the elongate tube), and/or in a reciprocating motion (e.g., backwards and forwards in a straight line along a longitudinal axis of the elongate tube). The distal portion of the elongate tube may be configured to swing from side-to-side in a vibrating or oscillatory motion. The distal portion of the elongate tube may be configured to move in a rotary motion (e.g., 360 degrees of rotation around a central axis of the elongate member).

In various embodiments, described here or otherwise within the scope of the present disclosure, an articulation wire and/or a conductive wire as a cutting wire may extend through or along a portion of the length or the entire length of the elongate tube and may be attached at or near the proximal end of the wire to an energy source.

In various embodiments, an external portion of the wire that is coextensive with a distal portion of the elongate tube, as described, may extend outside of the elongate tube and may be configured to extend at an outward angle relative to a longitudinal axis of the elongate tube as the distal portion bends or flexes. With the distal portion of the elongate tube held in a bent or flexed position, the medical professional may activate the energy source to energize the conductive wire and/or an articulation wire, thereby providing cutting and/or cauterizing energy that may, e.g., enlarge or further enlarge an opening of the target body lumen.

In various embodiments, the distal portion of the elongate tube and/or the distal end of one or more wires may be configured to deliver real-time feedback information (e.g., an electrical resistance, tissue temperature and/or impedance, a force of the device against a tissue, an activation time, a current, etc.) to a medical professional or computer processor, to manually or automatically adjust, e.g., increase or decrease, the frequency, power, and/or duration of energy being delivered. In any of the embodiments described or otherwise, a handle at the proximal end of the elongate tube and connected to a proximal end of the articulation wire may be actuatable to slide the articulation wire within a lumen such that the distal end of the tube is also articulated.

In various embodiments, fluoroscopy may assist in positioning a system or medical device according to the present disclosure, or for confirming the location of lumens, tissues, ducts, presence of one or more bodies such as gallstones, etc. A contrast agent may be injected through a medical device and into or about the body lumen for performing fluoroscopy. A contrast agent may be injected through a lumen of the medical device. A guidewire may be withdrawn from a lumen to allow the contrast agent to be injected through the lumen. Alternatively, a contrast agent may be injected in a lumen containing a guidewire such that the contrast agent flows through the lumen about the guidewire. Alternatively, a lumen or a portion of a multi-lumen (e.g., a bifurcated lumen) may be dedicated to the flow of a contrast agent and may be used to deliver contrast agent out of a medical device. Alternatively, a lumen may be used for a first purpose and later be used for a second purpose, e.g., first to receive a guidewire and second to flow a contrast agent. The contrast agent may comprise iodine, barium sulfate, gadolinium, or the like, or some combination thereof.

In various embodiments, described here or otherwise within the scope of the present disclosure, a portion of a lumen, e.g., the articulation wire lumen, may be a C-shaped channel extending along a portion of the flexible elongate tube where a portion of the articulation wire may extend outside of the tube. The channel may comprise other shapes such as, e.g., U-shaped, V-shaped, triangular, boxed, a combination of these shapes, etc. A channel, such as these channels, may be an open channel or a closed channel. A closed channel may have one or more portions of varying wall thickness, e.g., a thinner wall at a portion of the channel configured for a user to remove an object (e.g., a guidewire) from the channel by pulling or tearing the guidewire through the thinner portion of the wall. A wall of a closed channel may be perforated, e.g., for easier removal of the guidewire.

In various of the embodiments, described here or otherwise within the scope of the present disclosure, a bifurcated lumen may include a partition wall along at least a portion of the bifurcated lumen that separates the lumen into two half-lumens. Each of the two half-lumens may not be in fluid communication with each other such that each one may accept different fluids or objects, e.g., a contrast agent and a guidewire. The proximal portion of the bifurcated lumen may transition to two independent lumens. In embodiments, a lumen may be divided into more than two half-lumens, e.g., three, four, or more lumens, which may be equal in dimension or have different dimensions. The partition wall may be destructible such that, e.g., a distal portion of the partition wall may be destroyed or removed such that the parts of the bifurcated lumen converge together into one lumen. The length of the partition wall that is destroyed or removed may be customizable by the user. For example, the partition wall may be at least partially destructible at the distal end of a tube. Converged lumens may allow for a smaller diameter lumen extending through the distal end of the elongate tube compared to the larger diameters of the two separate lumens or half-lumens, allowing in some cases for more space at the distal end for other of the lumens. The separate half-lumens or lumens extending throughout most of the elongate tube may allow for multiple fluids to travel independently along most of the length of the elongate tube and exit the distal end of the tube, without the multiple fluids contacting each other along the length (as may be the case with fluids sharing a lumen along most of the length of the tube).

In various embodiments, methods of accessing an opening of a body lumen may include extending an endoscope having a working channel into a patient. A distal end of the elongate tube may be articulated toward the opening of the body lumen opening via a articulation wire that may extend through the elongate tube. The articulation wire may be connected at the distal end. The articulation wire may have a portion external to the elongate tube. The articulation may be slidable relative to the tube to articulate the distal end of the tube toward the opening. The body lumen may be cannulated with a distal end of the elongate tube. An electrode at a distal end of the elongate tube may be activated to cut a tissue of the opening. A guidewire may be extended through the elongate tube into the body lumen. The elongate tube may be removed from about the guidewire and the working channel such that the guidewire may be left within the working channel and the body lumen. Another instrument may be inserted within the working channel over the guidewire and into the body lumen. Prior to or during activation, a distal portion of the elongate tube may be oriented such that a portion of the electrode makes contact with the tissue of the body lumen. A contrast agent may be delivered into the body lumen.

The medical devices of the present disclosure are not limited to duodenoscopes, and may include a variety of medical devices for accessing body passageways, including, for example, catheters, ureteroscopes, bronchoscopes, colonoscopes, arthroscopes, cystoscopes, hysteroscopes, and the like.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A medical device, comprising:
a flexible elongate tube having a proximal end, a distal end, an outer surface, and a distal portion proximal to the distal end, the distal end configured to access an opening of a body lumen;
a first lumen extending from the distal end of the tube at least partially along the tube toward the proximal end of the tube, the first lumen configured to accept a guidewire;
a second lumen extending at least partially along the tube from the proximal end of the tube;
an articulation wire extending along the second lumen, a distal end of the articulation wire connected to the distal portion of the elongate tube, and a portion of the articulation wire, which is proximal to the distal end of the articulation wire, extending external to the outer surface of the elongate tube;
an electrode disposed on the distal portion of the tube distal to the distal end of the articulation wire; and
a conductive wire extending along and within the tube, the conductive wire in electrical communication with the electrode.

2. The medical device of claim 1, wherein the electrode extends linearly on the outer surface of the tube from the distal portion to an opening of the first lumen at the distal end of the tube.

3. The medical device of claim 1, wherein the electrode extends in a loop on the outer surface of the tube from the distal portion around the first lumen.

4. The medical device of claim 1, wherein the electrode comprises 304 stainless steel, aluminum, silver, or titanium.

5. The medical device of claim 1, wherein the electrode is a separate element disposed on the outer surface of the tube along the distal portion and coupled to the conductive wire.

6. The medical device of claim 1, further comprising an anchor within the distal portion of the tube and coupled to the distal end of the articulation wire.

7. The medical device of claim 6, wherein the second lumen extends to the distal portion of the tube, and the anchor is disposed within the second lumen.

8. The medical device of claim 1, further comprising a handle at the proximal end of the elongate tube and connected to the articulation wire, wherein the handle is actuatable to slide the articulation wire within the second lumen and articulate the distal end of the tube toward the opening of the body lumen.

9. The medical device of claim 1, wherein the articulation wire proximal and distal to the external portion is disposed within the second lumen.

10. The medical device of claim 1, wherein the articulation wire is slidably translatable within the second lumen to articulate the distal end of the tube.

11. The medical device of claim 1, wherein the articulation wire and the conductive wire are the same wire.

12. The medical device of claim 1, wherein the electrode has a surface area on the outer surface of the tube along the distal portion and has a circle, oval, square, or rectangular shape.

13. A medical device, comprising:
a flexible elongate tube having a proximal end, a distal end, an outer surface, and a distal portion proximal to the distal end, the distal end configured to access an opening of a body lumen;
a first lumen extending from the distal end of the tube at least partially along the elongate tube toward the proximal end of the elongate tube, the first lumen configured to accept a guidewire;
a second lumen extending at least partially along the tube from the proximal end of the tube;
an electrode disposed on the distal portion of the tube; and
an articulation wire extending along the second lumen, a distal end of the articulation wire in electrical communication with the electrode, and a portion of the articulation wire, which is proximal to the distal end of the articulation wire, extending external to the outer surface of the elongate tube.

14. The medical device of claim 13, further comprising an insulative layer disposed on the external portion of the articulation wire.

15. The medical device of claim 13, further comprising an anchor disposed in the second lumen along the distal portion, wherein the anchor and the electrode are a continuous unibody.

16. The medical device of claim 13, wherein the electrode extends linearly on the outer surface of the tube from the distal portion to an opening of the first lumen at the distal end of the tube.

17. A method of accessing an opening of a body lumen, comprising:
inserting an elongate tube of a medical device having a distal end into a patient to the opening of the body lumen;
articulating the distal end of the elongate tube toward the opening of the body lumen via an articulation wire extending through the elongate tube, the wire connected at the distal end of the tube and having a portion external to an outer surface of the elongate tube that is slidable relative to the tube to articulate the distal end toward the opening; and
activating an electrode on the outer surface at the distal end of the elongate tube to cut tissue of the opening.

18. The method of claim 17, wherein the electrode is an extension of the articulation wire.

19. The method of claim 17, wherein the electrode is a separate element in electrical communication with a conductive wire extending along the tube.

20. The method of claim 17, further comprising orienting the distal end of the elongate tube such that a portion of the electrode makes contact with the tissue of the body lumen.
